SEQUENCE LISTING (4 sequences)

SEQ ID NO: 1 - DNA, 4035 bp, Homo Sapiens
aattcaccaccatggtcgggctccttttgttttttttcccagcgatctttttggaggtgtcccttctccccagaagccccggcaggaaagtgttgctggcaggagcgtcgtctcagcgctcggtggccagaatggacggagatgtcatcattggagccctcttctcagtccatcaccagcctccggccgagaaagtgcccgagaggaagtgtggggagatcagggagcagtatggcatccagagggtggaggccatgttccacacgttggataagatcaacgcggacccggtcctcctgcccaacatcaccctgggcagtgagatccgggactcctgctggcactcttccgtggctctggaacagagcattgagttcattagggactctctgatttccattcgagatgagaaggatgggatcaaccggtgtctgcctgacggccagtccctccccccaggcaggactaagaagcccattgcgggagtgatcggtcccggctccagctctgtagccattcaagtgcagaacctgctccagctcttcgacatcccccagatcgcttattcagccacaagcatcgacctgagtgacaaaactttgtacaaatacttcctgagggttgtcccttctgacactttgcaggcaagggccatgcttgacatagtcaaacgttacaattggacctatgtctctgcagtccacacggaagggaattatggggagagcggaatggacgctttcaaagagctggctgcccaggaaggcctctgtatcgcccattctgacaaaatctacagcaacgctggggagaagagctttgaccgactcttgcgcaaactccgagagaggcttcccaaggctagagtggtggtctgcttctgtgaaggcatgacagtgcgaggactcctgagcgccatgcggcgccttggcgtcgtgggcgagttctcactcattggaagtgatggatgggcagacagagatgaagtcattgaaggttatgaggtggaagccaacgggggaatcacgataaagctgcagtctccagaggtcaggtcatttgatgattatttcctgaaactgaggctggacactaacacgaggaatccctggttccctgagttctggcaacatcggttccagtgccgccttccaggacaccttctggaaaatcccaactttaaacgaatctgcacaggcaatgaaagcttagaagaaaactatgtccaggacagtaagatggggtttgtcatcaatgccatctatgccatggcacatgggctgcagaacatgcaccatgccctctgccctggccacgtgggcctctgcgatgccatgaagcccatcgacggcagcaagctgctggacttcctcatcaagtcctcattcattggagtatctggagaggaggtgtggtttgatgagaaaggagacgctcctggaaggtatgatatcatgaatctgcagtacactgaagctaatcgctatgactatgtgcacgttggaacctggcatgaaggagtgctgaacattgatgattacaaaatccagatgaacaagagtggagtggtgcggtctgtgtgcagtgagccttgcttaaagggccagattaaggttatacggaaaggagaagtgagctgctgctggatttgcacggcctgcaaagagaatgaatatgtgcaagatgagttcacctgcaaagcttgtgacttgggatggtggcccaatgcagatctaacaggctgtgagcccattcctgtgcgctatcttgagtggagcaacatcgaatccattatagccatcgccttttcatgcctgggaatccttgttaccttgtttgtcaccctaatctttgtactgtaccgggacacaccagtggtcaaatcctccagtcgggagctctgctacatcatcctagctggcatcttccttggttatgtgtgcccattcactctcattgccaaacctactaccacctcctgctacctccagcgcctcttggttggcctctcctctgcgatgtgctactctgctttagtgactaaaaccaatcgtattgcacgcatcctggctggcagcaagaagaagatctgcacccggaagcccaggttcatgagtgcctgggctcaggtgatcattgcctcaattctgattagtgtgcaactaaccctggtggtaaccctgatcatcatggaaccccctatgcccattctgtcctacccaagtatcaaggaagtctaccttatctgcaataccagcaacctgggtgtggtggcccctttgggctacaatggactcctcatcatgagctgtacctactatgccttcaagacccgcaacgtgcccgccaacttcaacgaggccaaatatatcgcgttcaccatgtacaccacctgtatcatctggctagcttttgtgcccatttactttgggagcaactacaagatcatcacaacttgctttgcagtgagtctcagtgtaacagtggctctggggtgcatgttcactcccaagatgtacatcattattgccaagcctgagaggaatgtccgcagtgccttcaccacctctgatgttgtccgcatgcatgttggcgatggcaagctgccctgccgctccaacactttcctcaacatcttccgaagaaagaaggcaggggcagggaatgccaattctaatggcaagtctgtgtcatggtctgaaccaggtggaggacaggtgcccaagggacagcatatgtggcaccgcctctctgtgcacgtgaagaccaatgagacggcctgcaaccaaacagccgtcatcaaacccctcactaaaagttaccaaggctctggcaagagcctgaccttttcagataccagcaccaagaccctttacaacgtagaggaggaggaggatgcccagccgattcgctttagcccgcctggtagcccttccatggtggtgcacaggcgcgtgccaagcgcggcgaccactccgcctctgccgccccacctgaccgcagaggagacccccctcttcctggccgaaccagccctccccaagggcttgccccctcctctccagcagcagcagcaaccccctccacagcagaaatcgctgatggaccagctccagggagtggtcagcaacttcagtaccgcgatcccggattttcacgcggtgctggcaggccccggtggtcccgggaacgggctgcggtccctgtacccgcccccgccacctccgcagcacctgcagatgctgccgctgcagctgagcacctttggggaggagctggtctccccgcccgcggacgacgacgacgacagcgagaggtttaagctcctccaggagtacgtgtatgagcacgagcgggaagggaacacggaagaagacgaactggaagaggaggaggaggacctgcaggcggccagcaaactgaccccggatgattcgcctgcgctgacgcctccgtcgcctttccgcgactcggtggcctcgggcagctcggtgcccagctccccagtgtccgagtcggtgctctgcacccctcccaacgtatcctacgcctctgtcattctgcgggactacaagcaaagctcttccaccctgtaagctcgacggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaat

SEQ ID NO: 2 - protein, 1194 aa, Homo Sapiens [sequence omitted]

SEQ ID NO: 3 - DNA, 3560 bp, Homo Sapiens
agcttccaccatggtccttctgttgatcctgtcagtcttacttttgaaagaagatgtccgtgggagtgcacagtccagtgagaggagggtggtggctcacatgccgggtgacatcattattggagctctcttttctgttcatcaccagcctactgtggacaaagttcatgagaggaagtgtggggcggtccgtgaacagtatggcattcagagagtggaggccatgctgcataccctggaaaggatcaattcagaccccacactcttgcccaacatcacactgggctgtgagataagggactcctgctggcattcggctgtggccctagagcagagcattgagttcataagagattccctcatttcttcagaagaggaagaaggcttggtacgctgtgtggatggctcctcctcttccttccgctccaagaagcccatagtaggggtcattgggcctggctccagttctgtagccattcaggtccagaatttgctccagcttttcaacatacctcagattgcttactcagcaaccagcatggatctgagtgacaagactctgttcaaatatttcatgagggttgtgccttcagatgctcagcaggcaagggccatggtggacatagtgaagaggtacaactggacctatgtatcagccgtgcacacagaaggcaactatggagaaagtgggatggaagccttcaaagatatgtcagcgaaggaagggatttgcatcgcccactcttacaaaatctacagtaatgcaggggagcagagctttgataagctgctgaagaagctcacaagtcacttgcccaaggcccgggtggtggcctgcttctgtgagggcatgacggtgagaggtctgctgatggccatgaggcgcctgggtctagcgggagaatttctgcttctgggcagtgatggctgggctgacaggtatgatgtgacagatggatatcagcgagaagctgttggtggcatcacaatcaagctccaatctcccgatgtcaagtggtttgatgattattatctgaagctccggccagaaacaaaccaccgaaacccttggtttcaagaattttggcagcatcgttttcagtgccgactggaagggtttccacaggagaacagcaaatacaacaagacttgcaatagttctctgactctgaaaacacatcatgttcaggattccaaaatgggatttgtgatcaacgccatctattcgatggcctatgggctccacaacatgcagatgtccctctgcccaggctatgcaggactctgtgatgccatgaagccaattgatggacggaaacttttggagtccctgatgaaaaccaattttactggggtttctggagatacgatcctattcgatgagaatggagactctccaggaaggtatgaaataatgaatttcaaggaaatgggaaaagattactttgattatatcaacgttggaagttgggacaatggagaattaaaaatggatgatgatgaagtatggtccaagaaaagcaacatcatcagatctgtgtgcagtgaaccatgtgagaaaggccagatcaaggtgatccgaaagggagaagtcagctgttgttggacctgtacaccttgtaaggagaatgagtatgtctttgatgagtacacatgcaaggcatgccaactggggtcttggcccactgatgatctcacaggttgtgacttgatcccagtacagtatcttcgatggggtgaccctgaacccattgcagctgtggtgtttgcctgccttggcctcctggccaccctgtttgttactgtagtcttcatcatttaccgtgatacaccagtagtcaagtcctcaagcagggaactctgctacattatccttgctggcatctgcctgggctacttatgtaccttctgcctcattgcgaagcccaaacagatttactgctaccttcagagaattggcattggtctctccccagccatgagctactcagcccttgtaacaaagaccaaccgtattgcaaggatcctggctggcagcaagaagaagatctgtaccaaaaagcccagattcatgagtgcctgtgcccagctagtgattgctttcattctcatatgcatccagttgggcatcatcgttgccctctttataatggagcctcctgacataatgcatgactacccaagcattcgagaagtctacctgatctgtaacaccaccaacctaggagttgtcactccacttggatacaatggattgttgattttgagctgcaccttctatgcgttcaagaccagaaatgttccagctaacttcaacgaggccaagtatatcgccttcacaatgtacacgacctgcattatatggctagcttttgtgccaatctactttggcagcaactacaaaatcatcaccatgtgtttctcggtcagcctcagtgccacagtggccctaggctgcatgtttgtgccgaaggtgtacatcatcctggccaaaccagagagaaacgtgcgcagcgccttcaccacatctaccgtggtgcgcatgcatgtaggggatggcaagtcatcctccgcagccagcagatccagcagcctagtcaacctgtggaagagaaggggctcctctggggaaaccttaagttccaatggaaaatccgtcacgtgggcccagaatgagaagagcagccgggggcagcacctgtggcagcgcctgtccatccacatcaacaagaaagaaaaccccaaccaaacggccgtcatcaagcccttccccaagagcacggagagccgtggcctgggcgctggcgctggcgcaggcgggagcgctgggggcgtgggggccacgggcggtgcgggctgcgcaggcgccggcccaggcgggcccgagtccccagacgccggccccaaggcgctgtatgatgtggccgaggctgaggagcacttcccggcgcccgcgcggccgcgctcaccgtcgcccatcagcacgctgagccaccgcgcgggctcggccagccgcacggacgacgatgtgccgtcgctgcactcggagcctgtggcgcgcagcagctcctcgcagggctccctcatggagcagatcagcagtgtggtcacccgcttcacggccaacatcagcgagctcaactccatgatgctgtccaccgcggcccccagccccggcgtcggcgccccgctctgctcgtcctacctgatccccaaagagatccagttgcccacgaccatgacgacctttgccgaaatccagcctctgccggccatcgaagtcacgggcggcgcgcagcccgcggcaggggcgcaggcggctggggacgcggcccgggagagccccgcggccggtcccgaggctgcggccgccaagccagacctggaggagctggtggctctcaccccgccgtcccccttcagagactcggtggactcggggagcacaacccccaactcgccagtgtccgagtcggccctctgtatcccgtcgtctcccaaatatgacactcttatcataagagattacactcagagctcctcgtcgttgtgatctagag

SEQ ID NO: 4 - protein, 1212 aa, Homo Sapiens
MVLLLILSVLLLKEDVRGSAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCNSSLTLKTHHVQDSKMGFVINAIYSMAYGLHNMQMSLCPGYAGLCDAMKPIDGRKLLESLMKTNFTGVSGDTILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPKGSMGNGGRATMSSSNGKSVTWAQNEKSSRGQHLWQRLSIHINKKENPNQTAVIKPFPKSTESRGLGAGAGAGGSAGGVGATGGAGCAGAGPGGPESPDAGPKALYDVAEAEEHFPAPARPRSPSPISTLSHRAGSASRTDDDVPSLHSEPVARSSSSQGSLMEQISSVVTRFTANISELNSMMLSTAAPSPGVGAPLCSSYLIPKEIQLPTTMTTFAEIQPLPAIEVTGGAQPAAGAQAAGDAARESPAAGPEAAAAKPDLEELVALTPPSPFRDSVDSGSTTPNSPVSESALCIPSSPKYDTLIIRDYTQSSSSL

(19) United States
(12) Patent Application Publication
(10) Pub. No.: US 2007/0292898 A1
(43) Pub. Date: Dec. 20, 2007

(54) Functional Screening Assay

(76) Inventor: Arjan Buist, Weert (NL)

Correspondence Address: Philip S. Johnson;Johnson & Johnson, One Johnson & Johnson Plaza, New Brunswick, NJ, 08933-7003, US

(21) Appl. No.: 11/630,173

(22) PCT Filed: Jun. 22, 2005
(86) PCT No.: PCT/EP05/52903

§ 371 Date: Dec. 19, 2006

(30) Foreign Application Priority Data
Jun. 25, 2004 (EP) 04076851.7

(51) Int. Cl. G01N 33/566; C12N 1/04; C12N 15/06; C12N 15/63; C12N 5/10; C12P 21/00

(52) U.S. Cl. 435/7.21; 435/243; 435/320.1; 435/325; 435/71.1

(57) ABSTRACT

In a first aspect the present invention provides an inducible expression vector encoding a metabotropic glutamate receptor. In particular a tetracycline inducible expression vector such as for example the commercially available pcDNA4/TO mammalian expression vector (Invitrogen, Carlsbad, Calif. USA) comprising the nucleotide sequence encoding for a member of the Group I mGluRs, in particular for the human mGluR1a (SEQ ID No1) or mGluR5 receptor (SEQ ID No.3). In a more preferred embodiment the inducible expression vector is selected from the tetracycline inducible expression plasmids hmGlu1a-pcDNA4/TO (FIG. 4) and hmGlu5a-pcDNA4/TO (FIG. 5).

In a second aspect, the present invention provides a cell line comprising any of the aforementioned inducible expression vectors. In particular the T-Rex-293 cells stably transfected with the tetracycline inducible expression plasmids hmGlu1a-pcDNA4/TO (FIG. 4) and hmGlu5a-pcDNA4/TO (FIG. 5) which where deposited at the Belgian Coordinated Collections of Microorganisms (BCCM) as T-Rex-293-hmGlu1a-pcDNA4/TO clone on Jun. 24, 2004. In a third aspect the present invention provides a method to identify compounds capability to modulate the activity of a metabotropic glutamate receptor said method comprising the steps of; contacting the aforementioned cell line with the compound to be tested, and determining the effect of said test compound on the metabotropic glutamate receptor activity. The effect on the metabotropic glutamate receptor activity is typically determined by assessing the change in intracellular calcium, in particular using a fluorescent dye such as for example fluo-3-AM. It is also an object of the present invention to provide a method to identify a compound capable to interact with a metabotrobic glutamate receptor, in particular with a Group I mGluR receptor, said method comprising the steps of contacting the cells according to the invention with the compounds to be tested under appropriate conditions and determining the binding of said test compounds to the cells.

The present invention provides a novel method to identify substances that are modulators of group I metabotropic glutamate receptors. The method of the present invention provides the development of a cell line that depending on the assay conditions can either be used in a functional assay to identify substances that modulate the activity of the group I metabotropic gluatamate receptors mGlu1 or mGlu5 or in binding assay to identify substances that interact with the aforementioned receptors.

### BACKGROUND OF THE INVENTION

Glutamate is the major excitatory neurotransmitter in the mammalian brain. Glutamate produces its effects on central neurons by binding to and thereby activating cell surface receptors. These receptors have been subdivided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors capable of activating a variety of intracellular second messenger systems following the binding of glutamate or other potent agonists including quisqualate and 1-aminocyclopentane-1,3-dicarboxylic acid (ACPD) (Schoepp et al., Trends Pharmacol. Sci. 11:508, 1990; Schoepp and Conn, Trends Pharmacol. Sci. 14:13, 1993).

Activation of different metabotropic glutamate receptor subtypes in situ elicits one or more of the following responses: activation of phospholipase C, increases in phosphoinositide (PI) hydrolysis, intracellular calcium release, activation of phospholipase D, activation or inhibition of adenylyl cyclase, increases and decreases in the formation of cyclic adenosine monophosphate (cAMP), activation of guanylyl cyclase, increases in the formation of cyclic guanosine monophosphate (cGMP), activation of phospholipase A2, increases in arachidonic acid release, and increases or decreases in the activity of voltage- and ligand-gated ion channels (Schoepp and Conn, Trends Pharmacol. Sci. 14:13, 1993; Schoepp, Neurochem. Int. 24:439, 1994; Pin and Duvoisin, Neuropharmacology 34:1, 1995).

Thus far, eight distinct mGluR subtypes have been isolated via molecular cloning, and named mGluR1 to mGluR8 according to the order in which they were discovered (Nakanishi, Neuron 13:1031, 1994, Pin and Duvoisin, Neuropharmacology 34:1, 1995; Knopfel et al., J. Med. Chem. 38:1417, 1995). Further diversity occurs through the expression of alternatively spliced forms of certain mGluR subtypes (Pin et al., PNAS 89:10331, 1992; Minakami et al., BBRC 199:1136, 1994). All of the mGluRs are structurally similar,.in that they are single subunit membrane proteins possessing a large amino-terminal extracellular domain (ECD) followed by seven putative transmembrane domain (7TMD) comprising seven putative membrane spanning helices connected by three intracellular and three extracellular loops, and an intracellular carboxy-terminal domain of variable length (cytoplasmic tail) (CT) (see, Schematic FIG. 1a).

The eight mGluRs have been subdivided into three groups based on amino acid sequence identities, the second messenger systems they utilize, and pharmacological characteristics (Nakanishi, Neuron 13:1031, 1994; Pine and Duvoisin, Neuropharmacology 34:1, 1995; Knopfel et al., J. Med. Chem. 38:1417, 1995). The amino acid identity between mGluRs within a given group is approximately 70% but drops to about 40% between mGluRs in different groups. For mGluRs in the same group, this relatedness is roughly paralleled by similarities in signal transduction mechanisms and pharmacological characteristics.

The Group I mGluRs comprise mGluR1, mGluR5 and their alternatively spliced variants. The binding of agonists to these receptors results in the activation of phospholipase C and the subsequent mobilization of intracellular calcium. For example, Xenopus oocytes expressing recombinant mGluR1 receptors have been utilized to demonstrate this effect indirectly by electrophysiological means (Masu et al., Nature 349:760, 1991; Pin et al., PNAS 89:10331, 1992). Similar results were achieved with oocytes expressing recombinant mGluR5 receptors (Abe et al., J. Biol. Chem. 267:13361, 1992; Minakami et al., BBRC 199:1136, 1994).

The agonist potency profile for Group I mGluRs is quisqualate>glutamate=ibotenate>(2S,1′S,2′S)-2carboxycyclopropyl)glycine (L-CCG-I) >(1S,3R)-1-aminocyclopentane-1,3-dicarboxylic acid (ACPD).

Quisqualate is relatively selective for Group I receptors, as compared to Group II and Group III mGluRs, but it also potently activates ionotropic AMPA receptors (Pin and Duvoisin, Neuropharmacology, 34:1, Knopfel et al., J. Med. Chem. 38:1417, 1995).

Attempts at elucidating the physiological roles of Group I mGluRs suggest that activation of these receptors elicits neuronal excitation. Various studies have demonstrated that ACPD can produce postsynaptic excitation upon application to neurons in the hippocampus, cerebral cortex, cerebellum, and thalamus as well as other brain regions. Evidence indicates that this excitation is due to direct activation of postsynaptic mGluRs, but it has also been suggested to be mediated by activation of presynaptic mGluRs resulting in increased neurotransmitter release (Baskys, Trends Pharmacol. Sci. 15:92, 1992; Schoepp, Neurochem. Int. 24:439, 1994; Pin and Duvoisin, Neuropharmacology 34:1). Pharmacological experiments implicate Group I mGluRs as the mediators of this excitation.

Other studies examining the physiological roles of mGluRs indicatet that metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363: 347 (1993); Bortolotto et at., Nature 368: 740 (1994); Aiba et al., Cell 79: 365 (1994); Aiba et al., Cell 79: 377 (1994). A role for mGluR activation in nociception and analgesia also has been demonstrated, in particular for Group I mGluRs. Substantial evidence has been accumulating suggesting the involvement of Group I mGluRs in nociceptive transmission. Group I receptor antagonism, by means of pharmacophores, receptor specific antibodies, or antisense probes, blocked hyperalgesia and allodynia in models of neuropathic pain suggesting that mGluR1 and mGluR5 antagonists may provide for a novel, efficacious chronic pain therapy (Role of Group I metabotropic glutamate receptors mGlu1 and mGlu5 in nociceptive signaling. A. S. J. Lesage, Current neuropharmacology in press).

In addition. mGluR activation has been suggested to play a modulatory role in a variety of other normal processes including synaptic transmission neuronal development apoptotic neuronal death, synaptic plasticity, spatial learning, olfactory memory, central control of cardiac activity, waking, motor control, and control of the vestibulo-ocular reflex. For reviews, see Nakanishi, Neuron 13: 1031 (1994); Pin et al., Neuropharmacology 34: 1; Knopfel et al., J. Med. Chem. 38: 1417 (1995).

Metabotropic glutamate receptors also have been suggested to play roles in a variety of pathophysiological processes and disease states affecting the CNS. These include stroke, head trauma, anoxic and ischemic injuries, hypoglycemia, epilepsy, and neurodegenerative diseases such as Alzheimer's disease. Schoepp et al., Trends Pharmacol. Sci. 14: 13 (1993); Cunningham et al., Life Sci. 54: 135 (1994); Hollman et al., Ann. Rev. Neurosci. 17: 31 (1994); Pin et al., Neuropharmacology 34: 1 (1995); Knopfel tal., J. Med. Chem. 38: 1417 (1995).

Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as analgesia, as neuroprotective agents or as anticonvulsants.

To date, the search for therapeutic agents which will bind and specifically modulate the function of the Group I mGluRs has been hampered by the unavailability of a screening assay that provides not only a reproducible and reliable output in a functional assay but which can also be deployed in a binding assay.

Notwithstanding the fact that PCT publication WO 94/29449 provides the nucleic acids encoding the human metabotropic glutamate receptors mGluR1, -2, -3 and mGluR5 as well as methods to identify compounds that bind to or modulate these receptors and the fact that several laboratories have constructed cell lines expressing metabotropic glutamate receptors which appear to function appropriately (Abe et al., J. Biol. Chem. 267:13361, 1992; Tanabe et al., Neuron 8:169, 1992; Aramori and Nakanishi, Neuron 8:757, 1992, Nakanishi, Science 258:597, 1992; Thomsen et al., Brain Res. 619:22, 1992; Thomsen et al., Eur. J. Pharmacol. 227:361, 1992; O'Haraet al., Neuron 11:41, 1993; Nakjima et al., J. Biol. Chem. 268:11868, 1993; Tanabe et al., J. Neurosci. 13:1372, 1993; Saugstad et al., Mol. Pharmacol. 45:367, 1994; Okamoto et al., J. Biol. Chem. 269:1231, 1994; Gabellini et al., Neurochem. Int. 24:533, 1994; Lin et al., Soc. Neurosci. Abstr. 20:468, 1994; Flor et al., Soc. Neurosci. Abstr. 20:468, 1994; Flor et al., Neuropharmacology 34:149, 1994).

Other reports have noted that expression of functional mGluR expressing cell lines is not predictable. For example, Tanabe et al., (Neuron 8:169, 1992) were unable to demonstrate functional expression of mGluR3 and mGluR4, and noted difficulty obtaining expression of native mGluR1 in CHO cells. Gabellini et al., (Neurochem. Int. 24:533, 1994) also noted difficulties with mGluR1 expression in HEK 293 cells and it is possible that some of these difficulties may be due to desensitization characteristics of these receptors. This rapid desensitisation upon agonist stimulation may in addition, adversely affect the viability of cell lines expressing these receptors and makes the use of native mGluRs for screening difficult. The inducible expression of the mGluR1a receptor in CHO cells using a Lac-repression system overcame many of the problems related to the desensitization characteristics of these receptors (M. S. Nash et al., J Neurochem., 2001, 77, 1664-1667) but still suffer from lower levels of transcription than occur with constitutively expressed vectors. Given the significance of density on receptor pharmacology (Hermans E et al., Br. J. Pharmacol., 1999, 126, 873-882) and the difference in pharmacological profile of the three goups of mGluRs it would however, be desirable to have a screening procedure for identifying molecules specifically affecting the activities of the different mGluRs using the native receptor proteins.

It is an object of the present invention to solve the aforementioned problem in the art.

### SUMMARY OF THE INVENTION

In a first aspect the present invention provides an inducible expression vector encoding a metabotropic glutamate receptor. In particular a tetracycline inducible expression vector such as for example the commercially available pcDNA4/TO mammalian expression vector (Invitrogen, Carlsbad, Calif. USA) comprising the nucleotide sequence encoding for the desired metabotropic glutamate receptor. In a preferred embodiment the inducible expression vector according to the present invention encodes for a member of the Group I mGluRs, in particular for the human mGluR1a (SEQ ID No1) or mGluR5 receptor (SEQ ID No.3). In a more preferred embodiment the inducible expression vector is selected from the tetracycline inducible expression plasmids hmGlu1a-pcDNA4/TO (FIG. 4) and hmGlu5a-pcDNA4/TO (FIG. 5).

In a second aspect, the present invention provides a cell line comprising any of the aforementioned inducible expression vectors. In particular the T-Rex-293 cells stably transfected with the tetracycline inducible expression plasmids hmGlu1a-pcDNA4/TO (FIG. 4) and hmGlu5a-pcDNA4/TO (FIG. 5) which where deposited at the Belgian Coordinated Collections of Microorganisms (BCCM) as T-Rex-293-hmGlu1a-pcDNA4/TO clone on Jun. 24, 2004 with the accession number LMBP 6156CB and as the T-Rex-293-hmGlu5a-pcDNA4/TO on Jun. 24, 2004 with the accession number LMBP 6157CB respectively.

In a third aspect the present invention provides a method to identify compounds capability to modulate the activity of a metabotropic glutamate receptor said method comprising the steps of;
contacting the aforementioned cell line with the compound to be tested and
determining the effect of said test compound on the metabotropic glutamate receptor activity.
   The effect on the metabotropic glutamate receptor activity is typically determined by assessing the change in intracellular calcium, in particular using a fluorescent dye such as for example fluo-3-AM.

It is also an object of the present invention to provide a method to identify a compound capable to interact with a metabotrobic glutamate receptor, in particular with a Group I mGluR receptor, said method comprising the steps of contacting the cells according to the invention with the compounds to be tested under appropriate conditions and determining the binding of said test compounds to the cells.

Alternatively, the aforementioned binding assays are performed on cellular extracts of the cells according to the invention, in particular on cellular membrane preparations of T-Rex-293-hmGlu1a-pcDNA4/TO and T-Rex-293-hmGlu5a-pcDNA4/TO which where deposited at the Belgian Coordinated Collection of Microorganism on Jun. 24, 2004 with the accession numbers LMPB 6156CB and LMBP 6157CB respectively.

This and further aspects of the present invention will be discussed in more detail hereinafter.

### BRIEF DESCRIPTION OF THE DRAWING

FIG. 1 Activation of hmGlu5a-pcDNA4/TO by glutamate. HEK cells were transiently transfected with various amounts of the hmGlu5a-pcDNA4/TO construct and were induced with different concentrations of tetracycline for 24 h. The FLIPR response was expressed as fold induction over the baseline set at 1.

FIG. 2 Dose-response curves of glutamate on un-induced T-REx-293 cells stably expressing hmGlu1a-pcDNA4/TO or hmGlu1a-pcDNA4/TO.

FIG. 3. Effect of tetracycline induction of mGlu1a expression and functional response. a: Westernblot of T-REx-293 cells (first lane) or T-REx-293 hmGlu1a-pcDNA4/TO clone 7 cells induced with different concentrations of tetracycline for 24 h (middle lanes) or not induced at all (last lane). b: Dose-response of tetracycline induced maximal glutamate response in a FLIPR assay on T-REx-293 hmGlu1a-pcDNA4/TO cells. The FLIPR response was expressed as fold induction over the baseline set at 1.

FIG. 4. Tetracycline inducible expression plasmid hmGlu1a-pcDNA4/TO

FIG. 5. Tetracycline inducible expression plasmid hmGlu5a-pcDNA4/TO

### DETAILED DESCRIPTION

The present invention is based on the finding that G-protein coupled receptors such as the metabotrobic glutamate receptors, and in particular the Group I mGluR receptors, have an optimal expression level when it comes to the use of the recombinant receptors in both functional and binding assays. In present pharmacological research compound library screening is typically performed using assay components that allow both functional screening, i.e. the capability of a compound to modulate the receptor activity as well as binding experiments, i.e the capability of a compound to interact with the receptor protein. These assay are preferably performed using the human recombinant material. With G-protein coupled receptors however, one is confronted with the problem that functional expression requires correct post-translational processing, including integration into the plasma membrane, and accordingly high protein expression levels do not automatically result in good functional expression. As shown in the examples hereinafter, even the use of an inducible expression system such as for example described in Hermans E. et al., 1998, J. Neurochem. 70:1772-1775, does not automatically result in a good binding and functional screening properties. See for example, M. S. Nash et al., 2001, J. Neurochem. 77, 1664-1667, who address the low-level expression typically observed with inducible vectors/constructs by pre-treatment with sodiumbutyrate. It was surprisingly found by the present inventors that overexpression of Group I mGluRs reduces the amount of functional receptors on the cell surface. It may be that with increasing levels of expression the receptors form aggregates, i.e. dimeric or multimeric complexes reducing the number of functional cell surface receptors. These complexes have been shown for most of the class C G-protein coupled receptors (GPCRs) and for GPCRs from other classes as well, although the role of this dimerization is still unclear. The negative correlation between receptor expression levels and the amount of functional cell surface receptors may be more general. This insight may help assay development for other GPCRs in the future. As shown in the experimental part hereinafter, best results were obtained using the T-Rex-293 cells stably transfected with a tetracycline inducible plasmid encoding the Group I metabotrobic glutamate receptors.

For the purposes of describing the present invention: human mGlu1a or mGluR1a as used herein refers to the human metabotrobic glutamate receptor subunit known as in Desia et al, 1995, Mol. Pharmacol. 48:648, the amino acid sequence (SEQ ID No.:2) of which can be found at SwissProt Accession no. Q13255, as well as to its mammalian orthologs. mGluR1a also refers to other mGluR receptor subunits that have minor changes in amino acid sequence from those described hereinbefore, provided those other mGluR receptor subunits have substantially the same biological activity as the subunits described hereinbefore. A mGluR subunit has substantially the same biological activity if it has an amino acid sequence that is at least 80% identical to, preferably at least 95% identical to, more preferably at least 97% identical to, and most preferably at least 99% identical to SEQ ID No.: 2

mGluR5 as used herein refers to the human mGluR receptor subunit known as MGR5 in Minakami et al., 1994, Biochem. Biophys. Res. Commun. 199:1136-1143, the amino acid sequence (Seq Id NO.: 4) of which can be found at SwissProt accession no. P41594 as well as to its mammalian orthologs. mGluR5 also refers to other mGluR receptor subunits that have minor changes in amino acid sequence from those described hereinbefore, provided those other mGluR receptor subunits have substantially the same biological activity as the subunits described hereinbefore. A mGluR subunit has substantially the same biological activity if it has an amino acid sequence that is at least 80% identical to, preferably at least 95% identical to, more preferably at least 97% identical to, and most preferably at least 99% identical to SEQ ID No.: 4

It is thus an object of the present invention to provide an inducible expression vector encoding for a metabotrobic glutamate receptor, in particular encoding for a Group I mGluR receptor, more preferably encoding mGlur1a or mGlur5. “An inducible expression vector” refers to a vector for heterologous expression of a gene encoding a polypeptide of interest under the control of an “inducible” expression element. The expression elements are elements provided for expression of the protein of interest at suitable levels and at convenient times. Any of a wide variety of known expression elements may be used, for example selected from the group consisting of promoters, operators and ribosome binding sites. In the “inducible” expression vector according to the invention, said expression elements are regulatable, i.e. inducible—that is, for example a promoter to which transcription factors, etc., can be made to bind at will using exogenous factors such as metals, temperature, hormones, etc. Examples of the inducible promoter include, for instance, lac, tac, trc, trp, araB, Pzt-1, λ P_{L}, and the like. The lac, tac and trc promoters can be induced by using isopropyl-1-thio-β-D-galactopyranoside (IPTG) ; the trp, araB and Pzt-1 promoters can be induced by using 3-indoleacrylic acid (IAA), L-arabinose and tetracycline, respectively; and the λP_{L} promoter can be induced at a high temperature (42° C.). Also usable is a T7 promoter, which is specifically and strongly transcribed by a T7 RNA polymerase. In a preferred embodiment of the present invention the inducible expression element is based on the tetracycline system. In said system the E. coli tet repressor is fused the Herpes simplex virus virion protein 16 acidic activation domain (VP16) to create a tetracycline-controlled transactivator that in the presence of tetracycline or derivatives, can induce expression from polynucleotides operably linked to tet operators.

“Functional mGluR receptor” refers to a mGluR receptor formed by expression of a Group I mGluR receptor, preferably mGluR1a or mGluR5 in a cell, wherein said cell does not normally express the mGluR receptor, where the functional mGluR receptor mediates at least one functional response when exposed to the mGluR receptor agonist glutamate. Examples of functional responses are: pigment aggregation in Xenopus melanophores, negative modulation of cAMP levels, coupling to inwardly rectifying potassium channels, mediation of late inhibitory postsynaptic potentials in neurons, changes in intracellular calcium concentrations, increase of inositol phosphate, MAPKinase activation, extracellular pH acidification, and other functional responses typical of G-protein coupled receptors. One skilled in the art would be familiar with a variety of methods of measuring the functional responses of G-protein coupled receptors such as the mGluR receptor (see, e.g., Lerner, 1994, Trends Neurosci. 17: 142-146 [changes in pigment distribution in melanophore cells]; Yokomizo et al., 1997, Nature 387: 620-624 [changes in cAMP or calcium concentration; chemotaxis]; Howard et al., 1996, Science 273: 974-977 [changes in membrane currents in Xenopus oocytes]; McKee et al., 1997, Mol. Endocrinol. 11: 415-423 [changes in calcium concentration measured using the aequorin assay]; Offermanns & Simon, 1995, J. Biol. Chem. 270: 15175-15180 [changes in inositol phosphate levels]). It is well within the competence of one skilled in the art to select the appropriate method of measuring functional responses for a given experimental system.

The term “compound”, “test compound”, “agent” or “candidate agent” as used herein can be any type of molecule, including for example, a peptide, a polynucleotide, or a small molecule that one whishes to examine for their capability to modulate mGlur receptor activity, and wherein said agent may provide a therapeutic advantage to the subject receiving it. The candidate agents can be administered to an individual by various routes, including, for example, orally or parenterally, such as intravenously, intramuscularly, subcutaneously, intraorbitally, intracapsularly, intraperitoneally, intrarectally, intracisternally or by passive or facilitated absorption through the skin, using for example a skin patch or transdermal iontophoresis, respectively. Furthermore the compound can be administered by injection, intubation or topically, the latter of which can be passive, for example, by direct application of an ointment, or active, for example, using a nasal spray or inhalant, in which case one component of the composition is an appropriate propellant. The route of administration of the compound will depend, in part, on the chemical structure of the compound. Peptides and polynucleotides, for example, are not particular useful when administered orally because they can be degraded in the digective tract. However, methods for chemically modifying peptides, for example rendering them less susceptible to degradation are well know and include for example, the use of D-amino acids, the use of domains based on peptidomimetics, or the use of a peptoid such as a vinylogous peptoid.

The agent used in the screening method may be used in a pharmaceutically acceptable carrier. See, e.g., Remington's Pharmaceutical Sciences, latest edition, by E. W. Martin Mack Pub. Co., Easton, Pa. which discloses typical carriers and conventional methods of preparing pharmaceutical compositions that may be used in conjunction with the preparation of formulations of the agents and which is incorporated by reference herein.

Cells

As already outlined above, the present invention provides a cell line stably transfected with an inducible expression vector that directs the expression of the mGluRs, in particular the Group I mGluR receptors mGluR1a and mGluR5 as defined hereinbefore. The cells will be chosen to be compatible with the said vector and may for example be bacterial, yeast, insect or mammalian. The host cells may be cultured under conditions for expression of the gene, so that the encoded polypeptide is produced. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others.

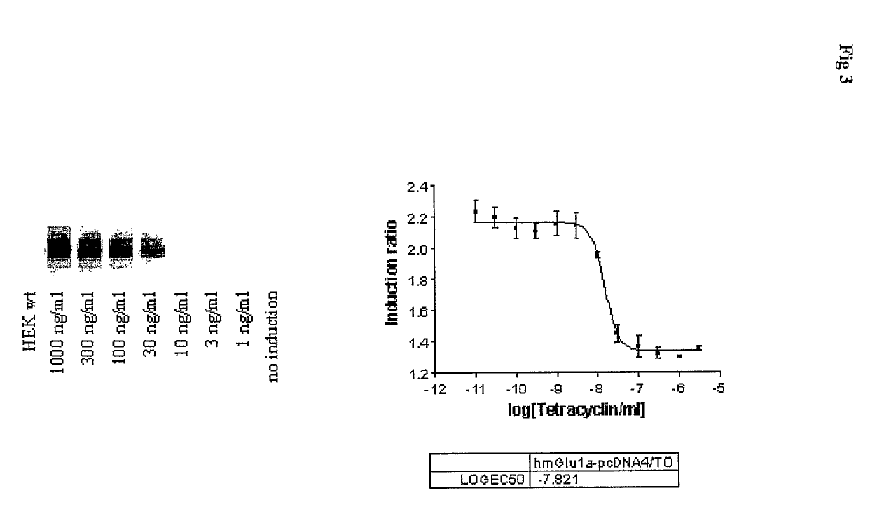

Depending on the cultivation conditions used the cells can either be used in a binding assay or in a functional screening assay. For functional expression of the mGluRs the cells are to be kept at uninduced to low induced cultivation conditions. “Unindunced” cultivation conditions as used herein refers to the absence of the exogenous factor that regulates the expression elements in the inducible vector, in the cultivation medium. In the object of the present invention uninduced cultivation conditions are to be used with inducible expression systems that have low leakiness as for example the interferon system, the Gal4-Estrogen Receptor system, the mutant Progesterone system, the mutant estrogen system and the Tetracycline system (described in table 2 of US patent application US 2003/0199022). “Low induced” cultivation conditions refer to the presence of the above metioned exogenous factors in the cultivation medium at a concentration which does not affect the functionality of the mGluR to be expressed. Based on the finding by the present inventors that decreasing the levels of mGluRs overexpression significantly increases the functional response, these low induction levels should result in cells that show no receptor binding and a strong functional response to agonist treatment. Actual methods to determine binding and the activity of mGluRs are known, see below, to those skilled in the art and accordingly used to determine the concentration of the exogenous factor in the cultivation medium, which does not affect the activity of the mGluRs to be expressed. See for example the effect of tetracycline induction on mGlu1a expression and functional response (FIG. 3) as described in the experimental part hereinafter. In this example the receptor binding is assessed using a radioligand binding assay, i.e. using [³H]R214127 as radioligand (Lavreysen et al., 2003) and the activity/functional response by measuring the change in intracellular calcium using a fluorescent indicator dye, in particular using fluo-3AM. For binding assays the inducible cells are to be kept under moderate to high induced cultivation conditions. “Moderate to high” cultivation conditions as used herein refers to the presence of the exogenous factor in the cultivation medium at a concentration that results in a Westernblot detectable expression of the mGluR receptors which does not result in a functional expression of the mGluRs.

It is accordingly an object of the present invention to provide the use of the aforementioned cells in;

i) a method for the functional expression of mGluRs, said method comprising the cultivation of the cells according to the invention at uninduced to low induced cultivation conditions, or

ii) an mGluR binding assay, said method comprising the cultivation of the cells according to the invention at moderate to high cultivation conditions.

In particular T-Rex-293 cells transfected with said expression vectors. Such expression vectors are routinely constructed in the art of molecular biology and may involve the use of plasmid DNA and appropriate initiators, promoters, enhancers and other elements, which may be necessary, and which are positioned in the correct orientation, in order to allow for protein expression. Generally, any system or vector suitable to maintain, propagate or express polynucleotides to produce a polypeptide in a host may be used. The appropriate nucleotide sequence, i.e. the polynucleotide sequences encoding either the human mGluR1a or mGluR5 subunit as defined hereinbefore, may be inserted into an expression system by any of a variety of well-known and routine techniques such as for example those set forth in Current Protocols in Molecular Biology, Ausbel et al. eds., John Wiley & Sons, 1997.

In a particular embodiment the T-Rex-293 cells according to the invention are transfected with the commercially available tetracycline inducible expression vectors pcDNA4/TO comprising the polynucleotide sequences encoding for human mGluR1a (SEQ ID No.:1) and human mGluR5 (SEQ ID No.: 3) respectively. More preferably the present invention provides the stably transfected cell lines T-Rex-293-hmGlu1a-pcDNA4/TO and T-Rex-293-hmGlu5a-pcDNA4/TO which where deposited at the Belgian Coordinated Collection of Microorganism on Jun. 24, 2004 with the accession numbers LMPB 6156CB and LMBP 6157CB respectively. Using the cell lines according to the invention, will not only allow a functional screening assay but also to perform, departing from the same cell line, a compound binding assay.

For further details in relation to the preparation of nucleic acid constructs, mutagenesis, sequencing, introduction of DNA into cells and gene expression, and analysis of proteins, see for example, Molecular Cloning: a Laboratory Manual: 2nd edition, Sambrook et al., 1989, Cold Spring Harbor Laboratory Press.

Assays

The present invention also provides an assay for a compound capable of interacting with a Group I mGluR receptor, which assay comprises: using the cell lines of the present invention, contacting said receptor with a putative binding compound; and determining whether said compound is able to interact with said receptor.

In one embodiment of the assay, the receptor or subunits of the receptor may be employed in a binding assay. Binding assays may be competitive or non-competitive. Such an assay can accommodate the rapid screening of a large number of compounds to determine which compounds, if any, are capable of binding to the polypeptides.

Within this context, the present invention provides a method to identify whether a test compound binds to an isolated mGluR receptor protein of the present invention, and is thus a potential agonist or antagonist of the mGluR receptor, said method comprising;

a) contacting cells comprising an inducible expression vector according to the invention and expressing a mGluR receptor, wherein such cells do not normally express said mGluR receptor, with the test compound in the presence and absence of a compound known to bind the mGluR receptor, and

b) determine the binding of the test compound to the mGluR receptor using the compound known to bind to the mGluR receptor as a reference.

Binding of the test compound or of the compound known to bind to the mGluR receptor, hereinafter also referred to as reference compound, is assessed using art-known methods for the study of protein-ligand interactions. For example, such binding can be measured by employing a labeled substance or reference compound. The test compound or reference compound can be labeled in any convenient manner known in the art, e.g. radioactively, fluorescently or enzymatically. In a particular embodiment of the aforementioned method, the compound known to bind to the mGluR receptor, also known as the reference compound is detectably labeled, and said label is used to determine the binding of the test compound to the mGluR receptor. Said reference compound being labeled using a radiolabel, a fluorescent label or an enzymatic label, more preferably a radiolabel. In a more particular embodiment, the present invention provides a method to identify whether a test compound binds to an isolated mGluR receptor protein, said method comprising the use of a compound known to bind to the mGluR receptor, wherein said reference compound is selected from glutamate, ACPD, CPCCOEt, AIDA, LY341495 or quisqualate, preferably selected from radiolabeld compounds known to bind to the mGluR receptor such as for example ³H-glutamate, ³H-CPCCOEt, ³H-AIDA or ³H-quisqualate.

Subsequently, more detailed assays can be carried out with those compounds found to bind, to further determine whether such compounds act as agonists or antagonists of the polypeptides of the invention. Examples of glutamate receptor assays are well known in the art, for example, see Aramori et al., Neuron 8:757 (1992); Tanabe et al., Neuron 8:169 (1992). The methodology described in those publications is incorporated herein by reference.

Thus, in a further embodiment the present invention provides a method to identify mGluR receptor agonists said method comprising,
a) contacting cells comprising an inducible expression vector according to the invention and expressing a mGluR receptor, wherein such cells do not normally express said mGluR receptor, with the test compound in the presence and absence of a mGluR receptor agonist, and
b) determine the binding of the labeled agonist to said cells,
   where if the amount of binding of the labeled agonist is less in the presence of the test compound, then the compound is a potential agonist of the mGluR receptor. As already specified for the general binding assay above, the binding of the mGluR receptor agonists is assessed using art-known methods for the study of protein-ligand interactions. The label is generally selected from a radioactive label, a fluorescent label or an enzymatic label, in particular a radiolabel wherein the agonist is selected from ³H-glutamate, ³H-ACPD or ³H-quisqualate.

Similarly, the present invention provides a method to identify mGluR receptor antagonists said method comprising,
a) contacting cells comprising an inducible expression vector according to the invention and expressing a mGluR receptor, wherein such cells do not normally express said mGluR receptor, with the test compound in the presence and absence of a mGluR receptor antagonist, and
b) determine the binding of the labeled antagonist to said cells,
   where if the amount of binding of the labeled antagonist is less in the presence of the test compound, then the compound is a potential antagonist of the mGluR receptor. As already specified for the general binding assay above, the binding of the mGluR receptor antagonists is assessed using art-known methods for the study of protein-ligand interactions. The label is generally selected from a radioactive label, a fluorescent label or an enzymatic label, in particular a radiolabel wherein the antagonist is selected from the group consisting of 7-(hydroxyimino)cyclopropa[b]chromen-1a-carboxylate ethyl ester (CPCCOEt) (Casabona et al. 1997; Hermans et al. 1998), LY3411495, Bay 367620 (Carroll et al. 2001, Mol. Pharmacol 59:965-973), NPS 2390 (Van Wagenen et al. 2000, Society for Neurocrine abstracts 618.3), MPEP (Sult et al. 1999, Br. J. Pharmacol 127:1097-1099), R214127 (Lavreysen et al. 2003, Mol. Pharmacol. 63:1082-1093) and (RS)-1-aminoindan-1,5-dicarboxylic acid (AIDA) (Moroni et al. 1997; Pellicciari et al. 1995).

In an alternative embodiment of the present invention, the aforementioned binding assays are performed on a cellular composition, i.e a cellular extract, a cell fraction or cell organels comprising a mGluR receptor as defined hereinbefore. More in particular, the aforementioned binding assays are performed on a cellular composition, i.e. a cellular extract, a cell fraction or cell organels comprising a mGluR receptor as defined hereinbefore, wherein said cellular composition, i.e. cellular extract, cell fraction or cell organels, is obtained from cells comprising an inducible expression vector according to the invention. More preferably, the cellular composition, i.e. cellular extract, cell fraction or cell organels, is obtained from the stably transfected cell lines T-Rex-293-hmGlu1a-pcDNA4/TO and T-Rex-293-hmGlu5a-pcDNA4/TO which where deposited at the Belgian Coordinated Collection of Microorganism on Jun. 24, 2004 with the accession numbers LMPB 6156CB and LMBP 6157CB respectively.

In a further embodiment the present invention provides a functional assay for identifying compounds that modulate the mGluR-recepor activity in the cells according to the invention. Such an assay is conducted using the cells of the present invention, i.e. transfected with an inducible expression vector encoding hmGlur1a or hmGluR5 resepctively. The cells are contacted with at least one reference compound wherein the ability of said compound to modulate the mGluR-receptor activity is known. Thereafter, the cells are contacted with a test compound and determined whether said test compound modulates the activity of the mGluR receptor compared to the reference compound. A “reference compound” as used herein refers to a compound that is known to bind and/or to modulate the mGluR receptor activity.

A compound or a signal that “modulates the activity” of a polypeptide of the invention refers to a compound or a signal that alters the activity of the polypeptide so that it behaves differently in the presence of the compound or signal than in the absence of the compound or signal. Compounds affecting modulation include agonists and antagonists. An agonist of the mGluR receptor encompasses a compound such as glutamate or quisqualate that activates mGluR receptor function. Alternatively, an antagonist includes a compound that interferes with mGluR receptor function. Typically, the effect of an antagonist is observed as a blocking of agonist-induced receptor activation. Antagonists include competitive as well as non-competitive antagonists. A competitive antagonist (or competitive blocker) interacts with or near the site specific for agonist binding. A non-competitive antagonist or blocker inactivates the function of the receptor by interacting with a site other than the agonist interaction site.

In one embodiment the present invention provides a method for identifying compounds that have the capability to modulate mGluR receptor activity, said method comprising;
a) contacting cells according to the invention with at least one reference compound, under conditions permitting the activation of the mGluR receptor;
b) contacting the cells of step a) with a test compound, under conditions permitting the activation of the mGluR receptor, and
c) determine whether said test compound modulates the mGluR receptor activity compared to the reference compound.

Methods to determine the capability of a compound to modulate the mGluR receptor activity are based on the variety of assays available to determine the functional response of G-protein coupled receptors (see above) and in particular on assays to determine the changes in calcium concentration, changes in cAMP formation and changes in inositol phosphate formation. Conditions permitting the activation of the mGluR receptor are generally known in the art, for example the cells according to the present invention may be used in a assay that measures intracellular calcium mobilization using a calcium sensitive fluorescent dye such as Fluo-3 or in an assay to determine the changes in ³H-inositol phosphate (³H-InsP) as previously described (Carruthers et al, 1997).

In the aforementioned assay an increase of intracellular calcium mobilization in the presence of the test compound is an indication that the compound activates the mGlur receptor activity, and accordingly that said test compound is a potential agonist of the mGluR receptor protein. A decrease of intracellular calcium mobilization in the presence of the test compound is an indication that the compound inactivates the mGluR receptor protein and accordingly that said test compound is a potential antagonist of the mGluR receptor protein.

Particularly preferred types of assays include binding assays and functional assays which may be performed as follows:

Using the inducible expression vectors of the present invention, together with the finding by the present inventors that there is a negative correlation between receptor expression levels and the amount of functional cell surface receptors, one can use cell lines stably transfected with the inducible expression vectors according to the present invention in either binding or functional assays depending on the induction level used. As outlined in the experimental part hereinafter, without induction or with only low induction the cells provide a functional expression of the mGluR receptors which is not detectable on a Westernblot. With moderate to high induction, the cells provide a Westernblot detectable expression of the mGluR receptors that can be used in binding assay but which does not result in a functional expression of the mGluR receptors.

Binding Assays

Over-expression of the mGluR receptor by induction of the cell lines according to the invention may be used to produce membrane preparations bearing said receptor for ligand binding studies. These membrane preparations can be used in conventional filter-binding assays (e.g. Using Brandel filter assay equipment) or in high throughput Scintillation Proximity type binding assays (SPA and Cytostar-T flashplate technology; Amersham Pharmacia Biotech) to detect binding of radio-labelled mGluR ligands (including ³H-glutamate, ³H-CPCCOEt, ³H-AIDA, ³H-R214127 or ³H-quisqualate) and displacement of such radio-ligands by competitors for the binding site. Radioactivity can be measured with Packard Topcount, or similar instrumentation, capable of making rapid measurements from 96-, 384-, 1536-microtitre well formats. SPA/Cytostar-T technology is particularly amenable to high throughput screening and therefore this technology is suitable to use as a screen for compounds able to displace standard ligands.

Another approach to study binding of ligands to mGluR protein in an environment approximating the native situation makes use of a surface plasmon resonance effect exploited by the Biacore instrument (Biacore). mGluR binding receptor in membrane preparations or whole cells could be attached to the biosensor chip of a Biacore and binding of ligands examined in the presence and absence of compounds to identify competitors of the binding site.

Functional Assays

mGluR receptors belong to the family G-protein coupled receptors that are coupled to the IP3 signalling pathway, which results in a release of intracellular Ca²⁺ upon activation of these receptors. This change in intracellular calcium may be measured in real time using a variety of techniques to determine the agonistic or antagonistic effects of particular compounds. Changes in intracellular calcium are measurable using several ion-sensitive fluorescent dyes, including fluo-3, fluo-4, fluo-5N, fura red and other similar probes from suppliers including Molecular Probes. The inhibition of calcium release as a result of mGluR receptor activation can thus be characterised in real time, using fluorometric and fluorescence imaging techniques, including fluorescence microscopy with or without laser confocal methods combined with image analysis algorithms.

Given the effect of mGluR receptor activation on K⁺ and Ca²⁺ channels (Kammermeier and Ikeda 1999 Neuron 22:819-829; Sharon et al 1997 J. Gen. Physiol. 109:447-490), in an alternative functional screen the modulatory effect of a compound is assessed through the changes in calcium and potassium fluxes. Therefore, recombinant mGluR receptor proteins functionally expressed in the cell lines of the present invention can be characterised using whole cell and single channel electrophysiology to determine the mechanism of action of compounds of interest. Electrophysiological screening, for compounds active at mGluR receptor proteins, may be performed using conventional electrophysiological techniques and when they become available, novel high throughput methods currently under development.

Another approach is a high throughput screening assay for compounds active as either agonists or modulators which affect calcium transients. This assay is based around an instrument called a FLuorescence Imaging Plate Reader ((FLIPR®), Molecular Devices Corporation). In its most common configuration, it excites and measures fluorescence emitted by fluorescein-based dyes. It uses an argon-ion laser to produce high power excitation at 488 nm of a fluorophore, a system of optics to rapidly scan the over the bottom of a 96-/384-well plate and a sensitive, cooled CCD camera to capture the emitted fluorescence. It also contains a 96-/4384-well pipetting head allowing the instrument to deliver solutions of test agents into the wells of a 96-/4384-well plate. The FLIPR assay is designed to measure fluorescence signals from populations of cells before, during and after addition of compounds, in real time, from all 96-/384-wells simultaneously. The FLIPR assay may be used to screen for and characterise compounds functionally active at the cell lines according to the invention.

A high throughput screening assay, specifically usefull to identify mGluR agonists/antagonists could consist of an arrangement wherein the stably transfected cell lines T-Rex-293-hmGlu1a-pcDNA4/TO and T-Rex-293-hmGlu5a-pcDNA4/TO which where deposited at the Belgian Coordinated Collection of Microorganism on Jun. 24, 2004 with the accession numbers LMPB 6156CB and LMBP6157CB respectively, are kept without or under low induction levels and loaded with an appropriate fluorescent dye, incubated with a test compound and after sufficient time to allow interaction (8-24 hours, typically 12-24 hours, in particular 24 hours.) the change in relative fluorescence units measured using an automated fluorescence plate reader such as FLIPR or Ascent Fluoroskan (commercially available from Thermo Labsystems, Brussel, Belgium).

This and other functional screening assays will be provided in the examples hereinafter.

Method of Treatment

A preferred use of the compounds identified using the methods of the present invention is in the treatment of neurological diseases and disorders. Patients suffering from a neurological disease or disorder can be diagnosed by standard clinical methodology. Neurological diseases or disorders include neuronal degenerative diseases, glutamate excitotoxicity, global and focal ischemic and hemorrhagic stroke, head trauma, spinal cord injury, hypoxia-induced nerve cell damage, and epilepsy. These different diseases or disorders can be further medically characterized. For example, neuronal degenerative diseases include Alzheimer's disease and Parkinson's disease.

Another preferred use of the compounds identified using the methods of the present invention is in the production of other therapeutic effects, such as analgesic effects, cognition-enhancement effects, or muscle relaxation effects. The compounds identified using the methods of the present invention are preferably used to produce one or more of these effects in a patient in need of such treatment.

Patients in need of such treatment can be identified by standard medical techniques. For example, the production of analgesic activity can be used to treat patients suffering from clinical conditions of acute and chronic pain including the following: preemptive preoperative analgesia; peripheral neuropathies such as occur with diabetes mellitus and multiple sclerosis; phantom limb pain; causalgia; neuralgias such as occur with herpes zoster; central pain such as that seen with spinal cord lesions; hyperalgesia; cancer pain and allodynia.

In a method of treating a patient, a therapeutically effective amount of a compound which in vitro modulates the activity of a chimeric receptor having at least the extracellular domain of a metabotropic glutamate receptor is administered to the patient. In particular the systemic or topical administration of an effective amount of a compound according to the invention, to warm-blooded animals, including humans. Typically, the compound modulates metabotropic glutamate receptor activity by acting as an allosteric modulator or as an agonist or antagonist of glutamate binding site activation. Preferably, the patient has a neurological disease or a disorder, preferably the compound has an effect on a physiological activity. Such physiological activity can be convulsions, neuroprotection, neuronal death, neuronal development, central control of cardiac activity, waking, control of movements and control of vestibo ocular reflex.

Diseases or disorders which can be treated by modulating metabotropic glutamate receptor activity include one or more of the following types: (1) those characterized by abnormal glutamate homeostasis; (2) those characterized by an abnormal amount of an extracellular or intracellular messenger whose production can be affected by metabotropic glutamate receptor activity; (3) those characterized by an abnormal effect (e.g., a different effect in kind or magnitude) of an intracellular or extracellular messenger which can itself be ameliorated by metabotropic glutamate receptor activity; (4) abnormal level of glutamate neuronal activity; (5) abnormal neuronal response upon mGluR activity mediated messengers and (6) other diseases or disorders in which modulation of metabotropic glutamate receptor activity will exert a beneficial effect, for example, in diseases or disorders where the production of an intracellular or extracellular messenger stimulated by receptor activity compensates for an abnormal amount of a different messenger.

The compounds and methods can also be used to produce other effects such as an analgesic effect, cognition-enhancement effect, and a muscle-relaxant effect.

A “patient” refers to a mammal in which modulation of an metabotropic glutamate receptor will have a beneficial effect. Patients in need of treatment involving modulation of metabotropic glutamate receptors can be identified using standard techniques known to those in the medical profession. Preferably, a patient is a human having a disease or disorder characterized by one more of the following: (1) abnormal glutamate receptor activity (2) an abnormal level of a messenger whose production or secretion is affected by metabotropic glutamate receptor activity; and (3) an abnormal level or activity of a messenger whose function is affected by metabotropic glutamate receptor activity.

By “therapeutically effective amount” is meant an amount of an agent which relieves to some extent one or more symptoms of the disease or disorder in the patient; or returns to normal either partially or completely one or more physiological or biochemical parameters associated with or causative of the disease.

More generally, this invention provides a method for modulating metabotropic glutamate receptor activity by providing to a cell having a metabotropic glutamate receptor an amount of a metabotropic glutamate receptor modulating molecule sufficient to either mimic one or more effects of glutamate at the metabotropic glutamate receptor, or block one or more effects of glutamate at the metabotropic glutamate receptor. The method can carried out in vitro or in vivo.

Such agents may be formulated into compositions comprising an agent together with a pharmaceutically acceptable carrier or diluent. The agent may in the form of a physiologically functional derivative, such as an ester or a salt, such as an acid addition salt or basic metal salt, or an N or S oxide. Compositions may be formulated for any suitable route and means of administration. Pharmaceutically acceptable carriers or diluents include those used in formulations suitable for oral, rectal, nasal, inhalable, topical (including buccal and sublingual), vaginal or parenteral (including subcutaneous, intramuscular, intravenous, intradermal, intrathecal and epidural) administration. The choice of carrier or diluent will of course depend on the proposed route of administration, which, may depend on the agent and its therapeutic purpose. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Gennaro et al., Remington's Pharmaceutical Sciences, Mack Publishing Company, Easton, Pa. 18th Edition, 1990.

The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium, carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

Throughout this description the terms “standard methods”, “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, NY 1989.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

Experimental

Material and Methods

Materials

T4 DNA ligase, and restriction endonucleases were obtained from Boehringer (Mannheim, Germany). Plasmid preparation kits and the Qiaquick PCR amplification kit were obtained from Qiagen (Hilden, Germany). The mammalian expression vector pcDNA3 and pcDNA4/TO, and T-REx-293 cells were obtained from Invitrogen (Carlsbad, Calif. USA). Dulbecco's modified Eagle medium (DMEM) with glutaMAX-I, foetal calf serum, and dialyzed foetal calf serum was obtained from Life Technologies (Gaithersburg, Md. USA). FuGENE 6 transfection reagent was obtained from Roche (Roche Molecular Biochemicals, Mannheim, Germany) and lipofectAMINE 2000 from Gibo-BRL (Eggenstein, Germany). CHO-dhfr-cells stably expressing rat mGlu1a and rat mGlu5a receptor were a kind gift from S. Nakanishi (Tokyo University, Tokyo, Japan).

Cloning of hmGlu1a and hmGlu5a in pcDNA4/TO

Both human mGlu1a and mGlu5a were cloned into the pcDNA4/TO mammalian expression vector (Invitrogen, Carlsbad, Calif. USA). In this vector, receptor expression is under the control of a CMV promoter and two tetracycline operators, which confers tetracycline-inducible expression on the insert. The full length reading frame from hmGlu1a was cut from the hmGlu1a-pMx clone described previously (Lavreysen et al., 2002) using SspI and EcoRI and was inserted into the pcDNA4/TO vector opened with EcoRI and EcoRV. The full length reading frame from hmGlu5a was cut from a hmGlu5a-pcDNA3 clone using HindIII and DraII and was inserted into the pcDNA4/TO vector opened with HindIII and DraII.

Cell Transfection and Culture

For transient transfection experiments the different expression plasmids were transfected in different cells using lipofectAMINE 2000 (Gibo-BRL, Eggenstein, Germany) according to the recommendations of the supplier. For permanent transfections the tetracycline inducible hmGlu1a-pcDNA4/TO and hmGlu5a-pcDNA4/TO expression plasmids were transfected using the FuGENE 6 reagent (Roche Molecular Biochemicals, Mannheim, Germany) into T-REx-293 cells stably expressing the Tet repressor (Invitrogen, Carlsbad, Calif. USA) according to the recommendations of the supplier. Monoclonal cell lines were isolated under Zeocin (200 μg/ml) and Blasticidin (5 μg/ml) in GlutaMax I medium supplemented with 10% heat inactivated dialyzed fetal calf serum and antibiotics (Life Technologies, Gaithersburg, Md. USA). The same medium was used for cell culturing. Zeocin and Blasticidin were left out at least 1 day before any assay. Receptor expression in cells transfected with the hmGlu1a-pcDNA4/TO or hmGlu5a-pcDNA4/TO construct was induced by treatment of the cells with 1000 ng/ml tetracyclin for 24 h unless indicated otherwise. Cell lines transfected with the hmGluR1a-pMx construct were induced with interferon-β for 24 h before the assays as previously described (Lavreysen et al., 2002).

Intracellular Calcium Response in hmGlu1a and hmGlu5a Receptor Expressing Cells Intracellular calcium ion levels ([Ca²⁺]ⱼ) were measured with the fluorescent indicator dye fluo-3-AM (Molecular Probes, Eugene, Oreg. USA) using the Fluorometric Imaging Plate Reader (FLIPR, Molecular Devices, Sunnyvale, Calif. USA). Cells transiently or permanently expressing the hmGlu1a or hmGlu5a receptor were seeded at 30,000 cells/well in Biocoat poly-D-lysine 96-wells black-clear bottom plates (Becton Dickinson, Bedford, UK) 24 h before the experiment. The day of the experiment, the cells were loaded for 1 h with 2 μM fluo-3-AM in culture medium at 37° C. in 95% air and 5% CO₂. Fluo-3-AM was dissolved in 10% pluronic acid/DMSO to facilitate loading of the dye into the cells. During the loading time, 5 mM probenecid, which inhibits P₄₅₀-glycoprotein mediated transport of the dye out of the cell, was present, and the pH of the medium was adjusted to 7.4 with 1 M HEPES. The cells were washed twice with 140 mM NaCl, 1 MM MgCl₂, 5 mM KCl, 10 mM glucose, 1.25 mM CaCl₂, 5 mM HEPES, 2.5 mM probenecid, pH 7.4 and were preincubated for 20 min at room temperature with buffer before measurement. Relative fluorescent units (RFU) were recorded for each well in function of time. The fluorescent peak obtained upon the addition of glutamate was taken as the signal. In each experiment, concentration response curves were run in triplicate.

Membrane Preparation

The membranes were prepared as total particulate fractions. The cell were cultured to 90% confluency on 145 mm petri dishes and induced with 1 μg/ml tetracycline for 24 h and treated with 5 mM sodium butyrate, 24 hours before collection. The cells were washed twice with ice cold phosphate buffered saline (PBS w/o Ca²⁺ and Mg²⁺), scraped from the plates in 50 mM Tris-HCl buffer, pH 7.4, and collected by centrifugation (10 minutes at 23,500 g at 4° C.). The cell pellet was homogenized in hypotonic 5 mM Tris-HCl buffer, pH 7.4. The homogenate was centrifuged at 30,000 g for 20 minutes at 4° C. The final pellet was homogenized in 50 mM Tris-HCl buffer, pH 7.4 and stored in aliquots at −70° C. A protein determination was performed using the Bradford protein assay (Bio-Rad, Hercules, Calif. USA) using bovine serum albumin (BSA) as standard.

Radioligand Binding

mGlu1 receptor binding. To characterize transiently and permanently hmGlu1a expressing cells we performed receptor binding assays using [³H]R214127 described previously (Lavreysen et al., 2003). In short, membranes were thawed on ice and re-homogenized in ice-cold binding buffer containing 50 mM Tris-HCl (pH 7.4), 1.2 mM MgCl₂, 2 mM CaCl₂. Ligand binding experiments were performed at apparent binding equilibrium (30 min incubation) with 20 μg membrane protein and 10 nM of radioligand. Non-specific binding was estimated in the presence of 1 μM R193845. The incubation was stopped by rapid filtration under suction over GF/C glass-fiber filters (Whatman, England) using a manual 40-well filtration manifold. The filters were then transferred to scintillation vials and, after the addition of Ultima-Gold MV, the radioactivity collected on the filters was counted in a Packard scintillation counter. mGlu5 receptor binding. For hmGlu5a receptor binding assays we used [3H]MPEP (Tocris, Essex, UK). Membranes were thawed on ice and re-homogenized in ice-cold binding buffer containing 50 mM Tris-HCl (pH 7.4), 1.2 mM MgCl₂, 2 mM CaCl₂. Ligand binding experiments were performed at apparent binding equilibrium (60 min incubation) with 40 μg membrane protein and 4 nM of radioligand. Non-specific binding was estimated in the presence of 10 μM MPEP. The incubation was stopped by rapid filtration under suction over GF/C glass-fiber filters (Whatman, England) using a manual 40-well filtration manifold. The filters were then transferred to scintillation vials and, after the addition of Ultima-Gold MV, the radioactivity collected on the filters was counted in a Packard scintillation counter.

Immunoblotting

For immunoblotting, 20 μg of membrane protein was subjected to SDS-PAGE (using a 3-8% Tris-Acetate gel, Invitrogen, Carlsbad, Calif. USA) and transferred to PVDF membrane (Amersham Pharmacia Biotech, Buckinghamshire, England) by semi-dry blotting (Bio-Rad, Hercules, Calif. USA) in NuPage transfer buffer (Invitrogen, Carlsbad, Calif. USA) supplemented with 10% methanol. To ensure that equivalent amounts of protein were loaded in each lane and that transfer was comparable, membranes were stained with Coomassie Stain solution (Bio-Rad, Hercules, Calif. USA) before immunoblotting. Blots were blocked for 1 hour with 5% non-fat dry milk/0.1% Tween 20 in PBS and incubated overnight with the first antibody in PBS containing 2.5% non-fat dry milk (for mGlu1a: AB1551 (Chemicon, Temecula, Calif. USA) diluted 1:200; for hmGlu5a: AB5232 (Chemicon, Temecula, Calif. USA) diluted 1:200). Subsequently, after extensive washing the blot was incubated with secondary antibody (peroxidase-conjugated anti-rabbit IgG from donkey) diluted 1:5000 in 0.1% Tween 20 in PBS. Detection was performed by using the chemiluminescence plus (ECL+) Western blotting analysis system (Amersham Pharmacia Biotech, Buckinghamshire, England).

Results

To develop in vitro tests for the human mGlu1a and mGlu5a receptors various cells were first transiently transfected with expression constructs of the hmGlu1a receptor. 48 hours after the transfections cell membranes were prepared and the expression of the hmGlu1a receptor was analyzed by immunoblot and by receptor binding as described in the materials and methods section. At the same time an aliquot of the cells was seeded in MW96 well plates and tested for calcium transients upon stimulation with glutamate in a functional FLIPR assay. A summary of this data is given in table I.

TABLE I
hmGlu1a receptor expression, binding and functional responses.

| | | | | | Western |
|---|---|---|---|---|---|
| Cell line | Construct | TB | Blanco | FLIPR | blot |
| HEK293 | hmGluR1a-pcDNA3 | 31369 | 647 | 130 | +++ |
| CHO | hmGluR1a-pcDNAS | 3019 | 944 | 116 | + |
| COS7 | hmGluR1a-pcDNA3 | 8800 | 565 | 100 | ++ |
| NIH3T3 | hmGluR1a-pcDNA3 | 434 | 405 | 100 | +/− |
| HeLa | hmGluR1a-pcDNA3 | 1092 | 447 | 140 | + |
| HeLa | hmGluR1a-pMx | 756 | 487 | 100 | +/− |
| L929 | hmGluR1a-pcDNA3 | 1681 | 414 | 100 | + |
| L929 | hmGluR1a-pMx | 731 | 552 | 260 | +/− |
| CHO | rat mGluR1a | 5010 | 415 | 320 | + |

Receptor expression based on the immunoblot is qualitatively expressed based on the intensity of the corresponding bands. The FLIPR responses are expressed as percentage stimulation of the baseline set at 100%. Receptor binding is qualitatively expressed based on the specific binding of the radioligand to the membranes, and the total binding (TB) and blanco is expressed in DPM.

Here we observed a strong correlation between the signal on a westernblot and receptor binding but no correlation with functional FLIPR response. Most strikingly, transiently transfected HEK cells showed strongest receptor expression based on the westernblot and exhibited the strongest binding, but these cells had only a very weak calcium response to glutamate. In contrast, L929 cells transiently transfected with the interfereon inducable construct hmGluR1a-pMx showed no binding and a very weak signal on a westernblot, but had a strong calcium response upon glutamate stimulation. A CHO cell line stably expressing rat mGlu1 exibeted a moderate receptor expression and receptor binding, but had a strong calcium response upon glutamate stimulation as well. Take together, these data suggested to us that hmGlu1a receptor overexpression like in the transiently transfected HEK cells interfered with receptor signaling.

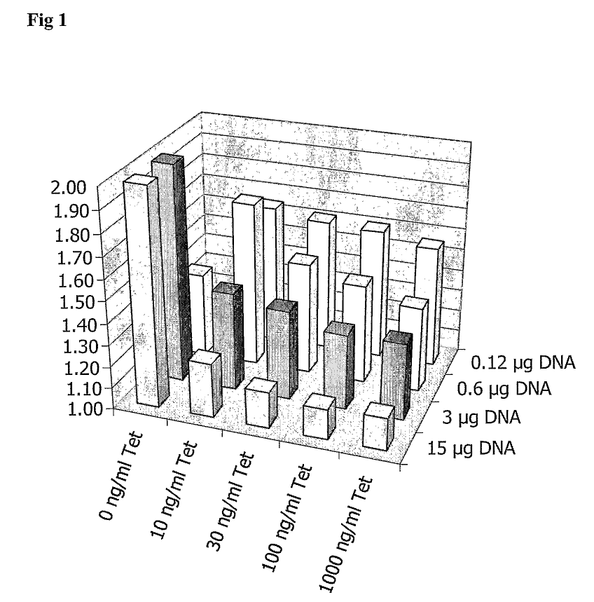

To further explore this hypothesis we cloned hmGlu1a and hmGlu5a in pcDNA4/TO which confers tetracycline-inducible expression of both receptors. T-REx-293 cells stably expressing the Tet repressor were transiently transfected with various amounts of the hmGlu5a-pcDNA4/TO construct. The cells were induced with different concentrations of tetracycline for 24 h. Subsequently, the cells were stimulated with glutamate and calcium released was assayed in a FLIPR (FIG. 1).

T-REx-293 cells transiently transfected with 15 or 3 μg hmGlu1a-pcDNA4/TO DNA showed the strongest Ca++ fluxes in response to glutamate when receptor expression was not induced with tetracycline. Apparently, the small receptor expression from promotor leakage yielded the highest number of functional receptors. Increasing receptor expression with tetracycline reduced the functional receptor responses. For T-REx-293 cells transiently transfected with 0.6 μg hmGlu1a-pcDNA4/TO DNA the strongest functional receptor responses were obtained after induction with a small amount of tetracycline that decreased again with increasing levels of tetracycline. This suggest that only moderate receptor expression levels will give maximum functional responses.

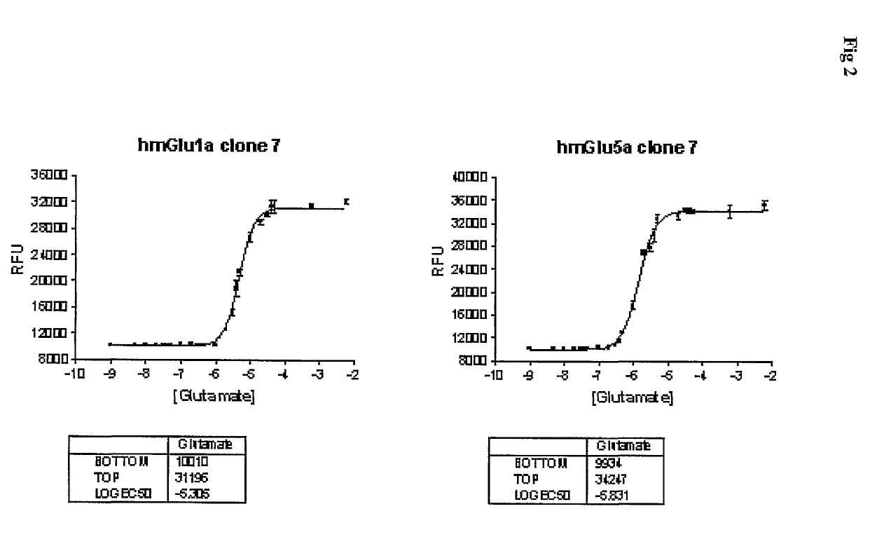

Next we made monoclonal cell lines of T-REx-293 cells stably expressing the hmGlu1a-pcDNA4/TO or hmGlu5a-pcDNA4/TO construct. Without any tetracycline treatment the cells were stimulated with various concentrations of glutamate and calcium released was assayed in a FLIPR (FIG. 2).

In addition, the T-REx-293 cells stably expressing hmGlu la-pcDNA4/TO were induced with different concentrations of tetracycline for 24 h. Cell membranes were prepared and the expression of the hmGlu1a receptor was analyzed by immunoblot (FIG. 3a). Only after induction with 3 ng/ml tetracycline for 24 h a faint signal could be detected. This signal increased with increasing amounts of tetracycline until a maximum level at 100 ng/ml tetracyclin.

At the same time an aliquot of the cells was seeded in MW96 well plates and tested for calcium transients upon stimulation with 1 mM glutamate in a functional FLIPR assay. The maximum FLIPR signal was plotted against the tetracycline concentration (FIG. 3b). The maximum FLIPR response was obtained in the absence of tetracycline induction and decreased after induction with more than 3 ng/ml tetracycline and was almost reduced to zero after 100 ng/ml tetracycline induction. In conclusion, the mGlu1a expression levels showed a very strong negative correlation with the functional receptor response.

Discussion

Previously an inducible stable human mGlu1a clone in L929 cells was developed in our lab that showed robust calcium fluxes in response to mGlu1a agonists eventhough receptor expression was extremely low as shown by westernblot analysis. In addition, no significant receptor binding could be establish with any of the available radioligands. Using transient transfections in HEK 293 we obtained strong mGlu1a receptor expression based on westernblot analysis and receptor binding studies. However, these cells showed only very weak functional receptor responses measured in a FLIPR assay. We hypothesized that overexpression of group I mGlu receptors interfered with receptor signaling. Here we demonstrate that decreasing the levels of receptor overexpression in HEK 293 cells increased the functional responses significantly. These results demonstrate that overexpression of group I mGlu receptors reduces the amount of functional receptors on the cell surface. Similar results were observed for mGlu5a. It may be that with increasing levels of expression the receptors form aggregates, i.e. dimeric or multimeric complexes reducing the number of functional cell surface receptors. These complexes have been shown for most of the class C GPCRs and for GPCRs from other classes as well, although the role of this dimerization is still unclear. The negative correlation between receptor expression levels and the amount of functional cell surface receptors may be more general. This insight may help assay development for other GPCRs in the future.

### REFERENCES

Carruthers , A. M., Challiss, R. A. J., and Pin, J.-P., Enhanced type 1α metabotropic glutamate receptor-stimulated phosphoinositide signaling after pertussis toxin treatment. Mol. Pharmacol. 52:406-414, 1997
Casabona, G., Knopfel, T., Kuhn, R., Gasparini, F., Baumann, P., Sortino, M. A., Copani, A., and Nicoletti, F. Expression and coupling to polyphosphoinositide hydrolysis of group I metabotropic glutamate receptors in early postnatal and adult rat brain. Eur. J. Neurosci. 1: 12-17, 1997.
Hermans, E., Nahorski, S. R., and Challiss, R. A. Reversible and non-competitive antagonist profile of CPCCOEt at the human type l metabotropic glutamate receptor. Neuropharmacology 37: 1645-1647, 1998
Lavreysen, H., Le Poul, E., Van Gompel, P., Dillen, L., Leysen, J. E., and Lesage, A. S. J. (2002) Supersensitivity of human metabotropic glutamate la receptor signaling in L929sA cells. Mol. Pharmacol. 61, 1244-1254.
Lavreysen, H., Janssen, C., Bischoff, F., Langlois, X., Leysen, J. E., and Lesage, A. S. J. (2003) [3H]R214127: A novel high-affinity radioligand for the mGlu1 receptor reveals a common binding site shared by multiple allosteric antagonist. Mol. Pharmacol. 63, 1082-1093.
Moroni, F., Lombardi, G., Thomsen, C., Leonardi, P., Attucci, S., Peruginelli, F., Torregrossa, S. A., Pellegrini-Giampietro, D. E., Luneia, R., and Pellicciari, R. Pharmacological characterization of 1-aminoindan-1,5-dicarboxylic acid, a potent mGluR1 antagonist. J. Pharmacol. Exp. Ther. 281: 721-729, 1997
Pellicciari, R., Luneia, R., Costantino, G., Marinozzi, M., Natalini, B., Jakobsen, P., Kanstrup, A., Lombardi, G., Moroni, F., and Thomsen, C. 1-Aminoindan-1,5-dicarboxylic acid: a novel antagonist at phospholipase C-linked metabotropic glutamate receptors. J. Med. Chem. 38: 3717-3719, 1995

1. A tetracycline inducible expression vector encoding for a metabotropic glutamate receptor.

2. A tetracycline inducible expression vector according to claim 1, wherein said vectors encodes for a member of the Group I mGluRs.

3. A tetracycline inducible expression vector according to claim 2, wherein said vector encodes for human mGluR1a (SEQ ID No:2) or human mGluR5 (SEQ ID No:4).

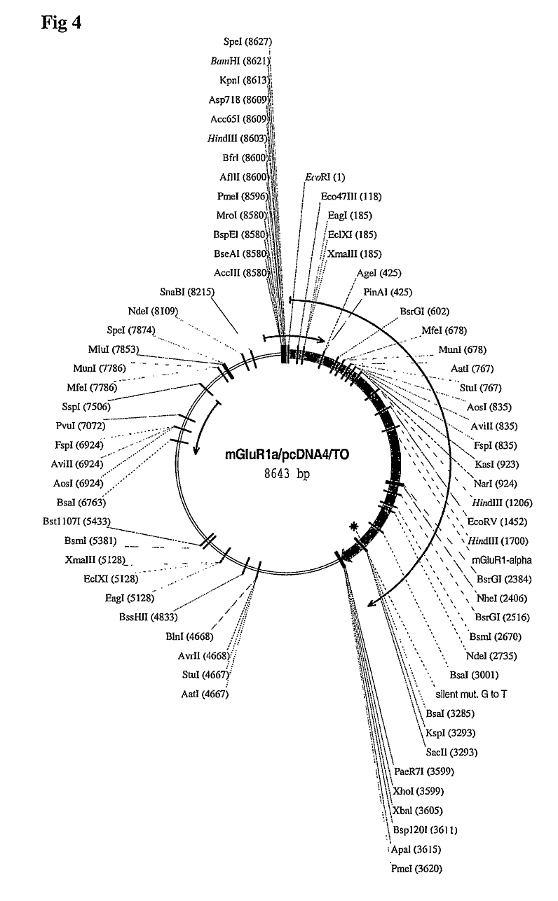

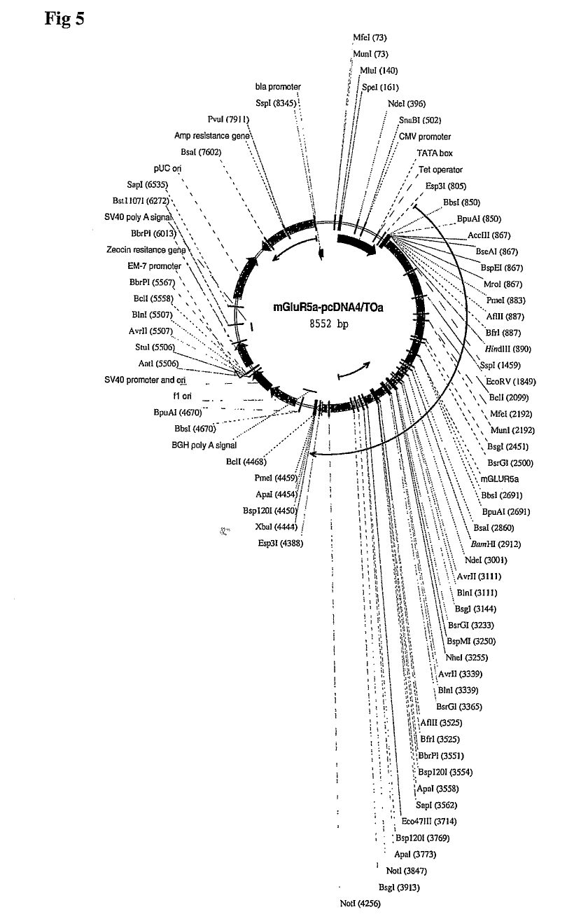

4. A tetracycline inducible expression vector according to claim 3, selected from the tetracycline inducible expression plasmids hmGlu1a-pcDNA4/TO (FIG. 4) and hmGlu5a-pcDNA4/TO (FIG. 5).

5. A host cell comprising a vector according to claim 1.

6. T-Rex-293 cells comprising a vector according to claim 1.

7. A method to identify whether a test compound binds to a mGluR receptor protein and is thus a potential agonist or antagonist of the mGluR receptor, said method comprising:
a) contacting cells comprising an inducible expression vector according to claim 1 and expressing a mGluR receptor, wherein such cells do not normally express the mGluR receptor, with the test compound in the presence and absence of a compound know to bind to the mGluR receptor, and
b) determine the binding of the test compound to the mGluR receptor using the compound known to bind to the mGluR receptor as a reference.

8. A method according to claim 7, wherein the compound known to bind to the mGluR receptor is detectably labeled, and wherein said label is used to determine the binding of the test compound to the mGluR receptor.

9. A method according to claim 8 wherein the compound known to bind to the MGLUR receptor is selected from the group consisting of ³H-glutamate, ³H-ACPD, and ³H-quisqualate.

10. A method according to claim 7 wherein the cells of step a) are kept at moderate to high induced cultivation conditions.

11. A method to identify compounds that have the capability to modulate mGluR receptor activity said method comprising,
a) contacting cells according to claim 5 with a least one reference compound under conditions permitting the activation of the mGluR receptor,
b) contacting the cells of step a) with a test compound under conditions permitting the activation of the mGluR receptor, and
c) determine whether said test compound modulates the mGluR receptor activity compared to the reference compound.

12. A method according to claim 11 wherein the reference compound is selected from the group consisting of glutamate, ACPD, CPCCCOEt, AIDA, LY341495 or quisqualate.

13. A method according to claim 11 wherein the cells of claim 5 are kept at uninduced or low induced cultivation conditions.

14. A method according to claim 11 wherein the capability of the test compound to modulate the mGluR receptor activity is determined using one or more of the functional responses selected form the group consisting of changes in cAMP, changes in calcium concentration, and changes in inositol phosphate levels.

15. A method according to claim 14 wherein the changes in intracellular calcium concentration are determined using ion-sensitive fluorescent dyes, including fluo-3, fluo-4, fluo-5N, fura red; in particular using fluo-3 AM.

16. A method according to claim 11 wherein the mGluR receptor agonist is selected from the group consisting of glutamate, ACPD or quisqualate.

17. Use of the cells according to claim 5 in;
a) a method for the functional expression of mGluRs, said method comprising the cultivation of the cells according to the invention at uninduced to low induced cultivation conditions, or
b i) an mGluR binding assay, said method comprising the cultivation of the cells according to the invention at moderate to high cultivation conditions.
